(19) United States
(12) Patent Application Publication
(10) Pub. No.: US 2024/0099690 A1
(43) Pub. Date: Mar. 28, 2024

(54) ULTRASONIC ENDOSCOPE

(71) Applicant: FUJIFILM Corporation, Tokyo (JP)

(72) Inventor: Hirotoshi KOSAWA, Kanagawa (JP)

(73) Assignee: FUJIFILM Corporation, Tokyo (JP)

(21) Appl. No.: 18/473,278

(22) Filed: Sep. 25, 2023

(30) Foreign Application Priority Data
Sep. 26, 2022 (JP) 2022-152752

(51) Int. Cl. A61B 8/12

(52) U.S. Cl. CPC A61B 8/12

(57) ABSTRACT

An ultrasonic endoscope includes a radial type ultrasound transducer in a distal end part of an insertion part, in which a distal end surface of the distal end part includes, when viewed from a direction perpendicular to the distal end surface, a center surface portion positioned on a center side of the distal end surface, and an annular outer peripheral surface portion positioned outside the center surface portion, an observation window, an illumination window, a forceps port, and a nozzle are provided in the center surface portion, the outer peripheral surface portion is a dead space of the distal end surface caused by the ultrasound transducer, and in a case where a longitudinal axis direction of the insertion part is defined as a height direction on the distal end surface, at least part of region of the outer peripheral surface portion has a height different from the center surface portion.

### CROSS-REFERENCE TO RELATED APPLICATION

The present application claims priority under 35 U.S.C § 119(a) to Japanese Patent Application No. 2022-152752 filed on Sep. 26, 2022, which is hereby expressly incorporated by reference, in its entirety, into the present application.

### BACKGROUND OF THE INVENTION

### 1. Field of the Invention

The present invention relates to an ultrasonic endoscope.

### 2. Description of the Related Art

In recent years, an ultrasonic endoscope is widely used for diagnosis and treatment in a medical field. As such an ultrasonic endoscope, an ultrasonic endoscope comprising a radial type ultrasound transducer is known (WO2020/188762A).

The ultrasonic endoscope shown in WO2020/188762A comprises an ultrasound transducer on an outer peripheral surface of a distal end part, and further comprises an observation optical system on a distal end surface of the distal end part. The ultrasound transducer irradiates an inside of a body of a subject with an ultrasonic wave and receives a reflected wave of the ultrasonic wave. The reflected wave is imaged. The observation optical system captures an image of the inside of the body of the subject.

### SUMMARY OF THE INVENTION

Because the radial type ultrasonic endoscope comprises the observation optical system, there is an increasing need for use in digestive tract observation. To enable digestive tract observation, the water droplet removal performance of the distal end surface, and in particular, an observation window becomes an important point of view.

Note that, because the radial type ultrasound transducer is disposed on the outer peripheral surface of the distal end part, an outer diameter of the insertion part increases. For this reason, a region of an outer periphery corresponding to the ultrasound transducer in the distal end surface of the distal end part becomes a dead space. On the other hand, the observation window and the illumination windows constituting the observation optical system, a nozzle for removing attachments to the observation window, and the like are disposed with bias to a center region of the distal end surface excluding the region of the outer periphery.

In the radial type ultrasonic endoscope having the above-described structure, because the outer diameter of the distal end surface increases in terms of the water droplet removal performance, water droplet and dirt are easily attached to the distal end surface. In addition, because the observation window and the nozzle are disposed with bias to the center, water droplet and dirt in the region of the outer periphery of the distal end surface is difficult to be removed. Furthermore, there is also a concern that water droplet and dirt in the center region of the distal end surface do not fly out even though being removed by the nozzle and return to the observation window and the like disposed in the center region.

The present invention has been accomplished in view of such a situation, and an object of the present invention is to provide a radial type ultrasonic endoscope capable of improving water droplet removal performance.

There is provided an ultrasonic endoscope of a first aspect comprising a radial type ultrasound transducer disposed in a distal end part of an elongated insertion part to be inserted into a subject, in which a distal end surface of the distal end part includes, in a case of being viewed from a vertical direction to the distal end surface, a center surface portion positioned on a center side of the distal end surface, and an annular outer peripheral surface portion positioned outside the center surface portion, an observation window, an illumination window, a forceps port, and a nozzle are provided in the center surface portion, the outer peripheral surface portion is a dead space of the distal end surface caused by the ultrasound transducer, and in a case where a longitudinal axis direction of the insertion part is defined as a height direction on the distal end surface, at least part of region of the outer peripheral surface portion has a height different from the center surface portion.

In an ultrasonic endoscope of a second aspect, the at least part of region includes a nozzle adjacent region adjacent to the nozzle.

In an ultrasonic endoscope of a third aspect, the nozzle adjacent region has a height lower than the center surface portion.

In an ultrasonic endoscope of a fourth aspect, the nozzle adjacent region includes a region from an extending position of a jetting port of the nozzle to a position of the nozzle on a side opposite to the observation window.

In an ultrasonic endoscope of a fifth aspect, the at least part of region includes an observation window adjacent region adjacent to the observation window.

In an ultrasonic endoscope of a sixth aspect, the observation window adjacent region has a height lower than the center surface portion.

In an ultrasonic endoscope of a seventh aspect, the observation window adjacent region has a height higher than the center surface portion.

In an ultrasonic endoscope of an eighth aspect, the observation window adjacent region is positioned on a side opposite to the nozzle with respect to the observation window.

In an ultrasonic endoscope of a ninth aspect, the observation window adjacent region is a region including a jetting range of a liquid jetted from the nozzle.

In an ultrasonic endoscope of a tenth aspect, the observation window adjacent region is a region including a range sandwiched between two virtual tangent lines in contact with an outer periphery of the observation window with a center of the jetting port of the nozzle as a starting point.

In an ultrasonic endoscope of an eleventh aspect, the at least part of region includes a nozzle adjacent region adjacent to the nozzle and an observation window adjacent region adjacent to the observation window.

In an ultrasonic endoscope of a twelfth aspect, the at least part of region is different in surface wettability from the center surface portion.

In an ultrasonic endoscope of a thirteenth aspect, the outer peripheral surface portion has a chamfer structure in an outer peripheral end portion.

In an ultrasonic endoscope of a fourteenth aspect, the outer peripheral surface portion is configured with a distal end cap that is a separate member from the center surface portion.

According to the present invention, it is possible to improve water droplet removal performance.

### BRIEF DESCRIPTION OF THE DRAWINGS

FIG. 1 is a schematic configuration diagram showing a configuration of an ultrasonography system using an ultrasonic endoscope.

FIG. 2 is an enlarged view showing an appearance of a distal end part of the ultrasonic endoscope shown in FIG. 1.

FIG. 3 is a sectional view of a distal end hard part of the ultrasonic endoscope shown in FIG. 1.

FIG. 4 is an enlarged plan view of a distal end surface of a first embodiment as viewed from a vertical direction.

FIG. 5 is a partial sectional view of the distal end hard part along the line 5-5 of FIG. 4.

FIG. 6 is a partial sectional view of a distal end hard part of Modification Example 2 of the first embodiment.

FIG. 7 is a partial sectional view of the distal end hard part taken along line 7-7 of FIG. 4.

FIG. 8 is an enlarged plan view of a distal end surface of a second embodiment as viewed from a vertical direction.

FIG. 9 is a partial sectional view of the distal end hard part taken along the line 9-9 of FIG. 8.

FIG. 10 is a partial sectional view of a distal end hard part of Modification Example 2 of the second embodiment.

### DESCRIPTION OF THE PREFERRED EMBODIMENTS

Hereinafter, an embodiment of an ultrasonic endoscope according to the present invention will be described referring to the accompanying drawings.

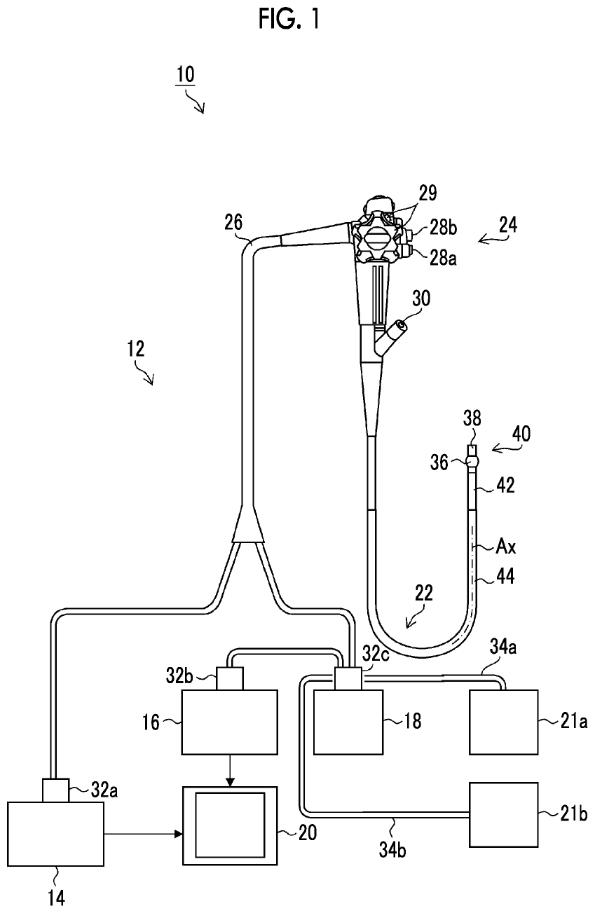

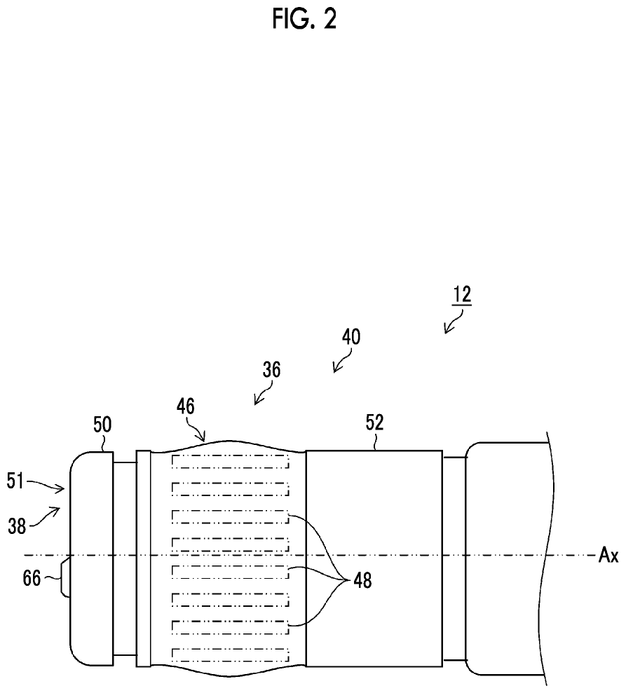

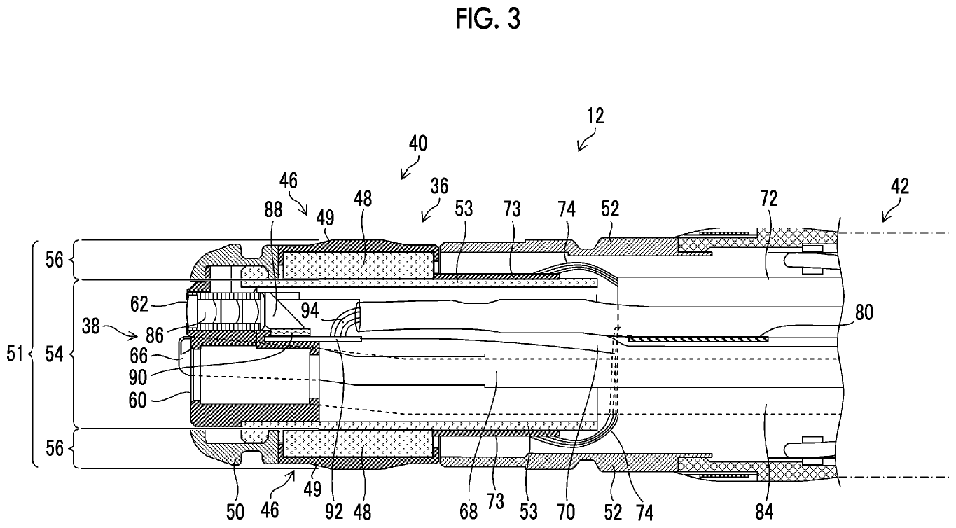

FIG. 1 is a schematic configuration diagram showing an example of an ultrasonography system 10 that uses an ultrasonic endoscope 12 of the embodiment. FIG. 2 is an enlarged view showing an appearance of a distal end part of the ultrasonic endoscope 12 shown in FIG. 1. FIG. 3 is a sectional view of the distal end hard part 40.

Ultrasonography System

As shown in FIG. 1, the ultrasonography system 10 comprises the ultrasonic endoscope 12, an ultrasound processor device 14 that generates an ultrasound image, an endoscope processor device 16 that generates an endoscope image, a light source device 18 that supplies illumination light, with which the inside of a body cavity is illuminated, to the ultrasonic endoscope 12, and a monitor 20 that displays the ultrasound image and the endoscope image. The ultrasonography system 10 comprises a water supply tank 21a that stores cleaning water or the like, and a suction pump 21b that sucks aspirates inside the body cavity.

The ultrasound processor device 14 generates and supplies an ultrasound signal for making the ultrasonic observation part 36 of the ultrasonic endoscope 12 generate an ultrasonic wave. The ultrasound processor device 14 receives and acquires an echo signal reflected from an observation target part irradiated with the ultrasonic wave, by the ultrasonic observation part 36 and executes various kinds of signal processing on the acquired echo signal to generate an ultrasound image.

The endoscope processor device 16 receives an image signal acquired from the observation target part illuminated with the illumination light from the light source device 18 in the endoscope observation part 38 of the ultrasonic endoscope 12. Then, the endoscope processor device 16 executes various kinds of signal processing and image processing on the acquired image signal to generate an endoscope image.

In the present example, the ultrasound processor device 14 and the endoscope processor device 16 are configured with two devices (computers) provided separately. Note that the present invention is not limited thereto, and both the ultrasound processor device 14 and the endoscope processor device 16 may be configured with one device.

The light source device 18 generates illumination light, such as white light consisting of light of three primary colors of red light, green light, and blue light or light of a specific wavelength. The illumination light propagates through the ultrasonic endoscope 12 and is emitted from the endoscope observation part 38, and the observation target part inside the body cavity is illuminated with the illumination light.

The monitor 20 receives respective video signals generated by the ultrasound processor device 14 and the endoscope processor device 16 and displays an ultrasound image and an endoscope image. In regard to the display of the ultrasound image and the endoscope image, only one image may be appropriately switched and displayed on the monitor 20 or both images may be displayed simultaneously.

In the present example, although the ultrasound image and the endoscope image are displayed on one monitor 20, a monitor for ultrasound image display and a monitor for endoscope image display may be provided separately. Alternatively, the ultrasound image and the endoscope image may be displayed in a display form other than the monitor 20, for example, in a form of being displayed on a display of a terminal carried with an operator.

Ultrasonic Endoscope

As shown in FIG. 1, the ultrasonic endoscope 12 has an elongated insertion part 22 that is inserted into the subject, an operating part 24 that is consecutively provided in a proximal end part of the insertion part 22 and is used by the operator to perform an operation, and a universal cord 26 that has one end connected to the operating part 24.

In the operating part 24, an air/water supply button 28a that opens and closes an air/water supply pipe line (not shown) from the water supply tank 21a, and a suction button 28b that opens and closes a suction pipe line (not shown) from the suction pump 21b are provided side by side. In the operating part 24, a pair of angle knobs 29 and a treatment tool insertion port 30 are provided.

In the other end portion of the universal cord 26, an ultrasound connector 32a that is connected to the ultrasound processor device 14, an endoscope connector 32b that is connected to the endoscope processor device 16, and a light source connector 32c that is connected to the light source device 18 are provided. The ultrasonic endoscope 12 is attachably and detachably connected to the ultrasound processor device 14, the endoscope processor device 16, and the light source device 18 through the connectors 32a, 32b, and 32c, respectively. The connector 32c is provided with an air/water supply tube 34a that is connected to the water supply tank 21a, and a suction tube 34b that is connected to the suction pump 21b.

The insertion part 22 has, in order from a distal end side, a distal end hard part 40 that has an endoscope observation part 38 and an ultrasonic observation part 36, a bendable part 42 that is connected to a proximal end side of the distal end hard part 40, and a soft part 44 that connects a proximal end side of the bendable part 42 and a distal end side of the operating part 24. The distal end hard part 40, the bendable part 42, and the soft part 44 are provided along a longitudinal axis Ax direction of the insertion part 22.

Next, the configuration of the distal end hard part 40 and a plurality of contents that are inserted into the insertion part 22 will be described referring to FIGS. 2 and 3. As shown in FIG. 2, the endoscope observation part 38 is provided in a distal end surface 51 of the distal end hard part 40. The endoscope observation part 38 acquires the endoscope image. The distal end hard part 40 is an example of a distal end part of an insertion part of the present invention. The ultrasonic observation part 36 is provided at a position on a proximal end side with respect to the distal end surface 51 in the distal end hard part 40. The distal end surface 51 is a portion on a side opposite to a side connected to the bendable part 42 in the distal end hard part 40.

The distal end hard part 40 has an annular distal end cap 50 that covers a distal end-side portion, and a proximal end-side ring 52 (also referred to as a balloon ring) that is disposed on a proximal end side of the ultrasonic observation part 36. The distal end cap 50 and the proximal end-side ring 52 are made of an insulating member, such as a hard resin, and serve as an exterior member. Though will be described below, the distal end cap 50 is an example of a member that constitutes an outer peripheral surface portion of the present invention.

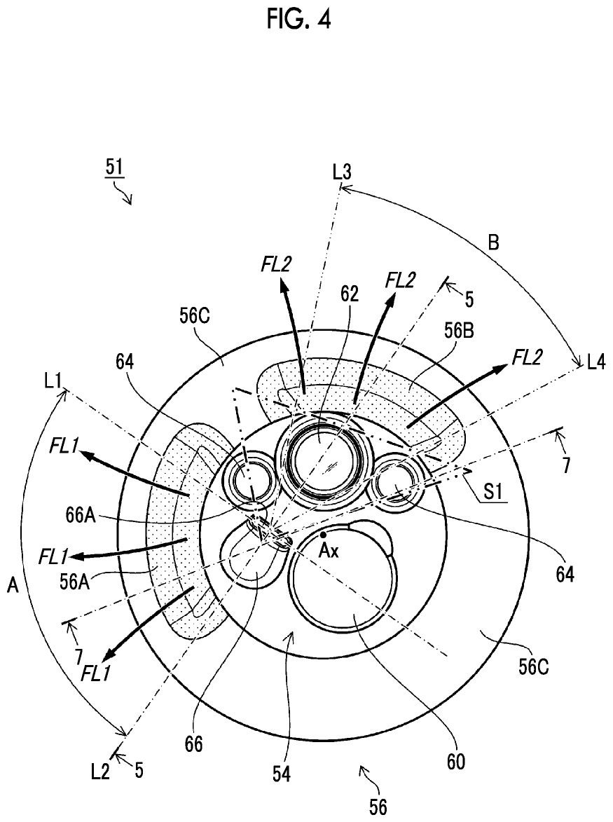

The distal end hard part 40 comprises a center surface portion 54 positioned on a center side of the distal end surface 51. An observation window 62 and illumination windows 64 that constitute the endoscope observation part 38 are disposed in the center surface portion 54. In the distal end surface 51, a nozzle 66 for removing attachments to the forceps port 60 and the observation window 62 is further disposed (see FIG. 4).

As shown in FIG. 3, the distal end hard part 40 comprises, for example, an observation unit in which a lens group 86, a prism 88, an imaging element 90, a substrate 92, and a plurality of signal lines 94 are disposed at the back (proximal end side) of the observation window 62.

Reflected light of the observation target part incident from the observation window 62 is taken in by the lens group 86. An optical path of the taken-in reflected light is folded at a right angle by the prism 88, and the reflected light forms an image on an imaging surface of the imaging element 90. The imaging element 90 photoelectrically converts the reflected light of the observation target part that has been transmitted through the observation window 62, the lens group 86, and the prism 88 has formed the image on the imaging surface, to output an image signal. Examples of the imaging element 90 include a charge coupled device (CCD) and a complementary metal oxide semiconductor (CMOS).

The imaging element 90 is mounted on the substrate 92. A circuit pattern that is electrically connected to the imaging element 90 is formed on the substrate 92. The circuit pattern comprises a plurality of electrodes in an end portion, and a plurality of signal lines 94 are connected to a plurality of electrodes, respectively. A plurality of signal lines 94 are bundled and are inserted into the operating part 24 from the bendable part 42 through the soft part 44 shown in FIG. 1 in a state of a shield cable. Then, a plurality of signal lines 94 are inserted into the universal cord 26 from the operating part 24 and are connected to the endoscope connector 32b.

The forceps port 60 disposed in the center surface portion 54 of the distal end surface 51 corresponds to an outlet of a forceps tube 84. The forceps tube 84 extends to a proximal end side of the insertion part 22 and extends to communicate with the treatment tool insertion port 30 of the operating part 24. A treatment tool, such as forceps, is inserted into the forceps tube 84 from the treatment tool insertion port 30 of the operating part 24 and protrudes from the forceps port 60. Treatment of the subject is performed by the treatment tool. That is, the forceps port 60 functions as a treatment tool outlet port.

The forceps tube 84 is connected to a negative pressure source (not shown). In a case where the suction button 28b is depressed, air is sucked from the forceps port 60 by way of the forceps tube 84 by the negative pressure source. With this, for example, cleaning water and a body liquid, such as residues (blood and the like), in the subject, are sucked from the forceps port 60 through the forceps tube 84. That is, the forceps port 60 functions as a suction port.

An emission end of a light guide 70 is connected to the illumination windows 64 (see FIG. 4). The light guide 70 is provided to extend from the insertion part 22 to the operating part 24. An incidence end of the light guide 70 is connected to the light source device 18 connected through the universal cord 26. The light guide 70 extends toward the proximal end side of the insertion part 22, is inserted into the universal cord 26 from the operating part 24, and is finally connected to the light source connector 32c. The light source connector 32c is connected to the light source device 18 (see FIG. 1). Illumination light emitted from the light source device 18 propagates through the light guide 70, and the part to be observed is irradiated with the illumination light from the illumination windows 64.

An air/water supply channel 68 is connected to the nozzle 66. The air/water supply channel 68 extends toward the proximal end side of the insertion part 22 and is inserted into the universal cord 26 from the operating part 24. The air/water supply channel 68 is connected to the light source connector 32c and is connected to the water supply tank 21a through the air/water supply tube 34a. To clean the surfaces of the observation window 62 and the illumination windows 64, the nozzle 66 jets air or cleaning water from the water supply tank 21a toward the observation window 62 and the illumination windows 64 through the air/water supply channel 68 in the ultrasonic endoscope 12.

As shown in FIG. 3, a cylindrical base member 53 (also referred to as a shield ring) is disposed on a proximal end side of the distal end cap 50. The ultrasound transducer 46 that constitutes the ultrasonic observation part 36 is disposed on an outer peripheral surface of the base member 53. The base member 53 has a function of shielding electromagnetic waves emitted from the ultrasound oscillators 48 of the ultrasound transducer 46, in addition to a function of supporting the ultrasound transducer 46.

The ultrasonic observation part 36 shown in FIG. 2 is configured with the ultrasound transducer 46. The ultrasound transducer 46 is configured as a radial type ultrasound transducer, and is configured by arranging a plurality of ultrasound oscillators 48 in a peripheral direction of the outer peripheral surface of the base member 53 shown in FIG. 3.

As shown in FIG. 2, the ultrasound transducer 46 is an array of a plurality of channels (CH) made of a plurality of ultrasound oscillators 48, for example, 48 to 192 rectangular parallelepiped ultrasound oscillators 48 arranged in a cylindrical shape. In the ultrasound transducer 46, as an example, a plurality of ultrasound oscillators 48 are arranged at predetermined pitches in a peripheral direction as in the example shown in the drawing. In this way, the ultrasound oscillators 48 constituting the ultrasound transducer 46 are arranged at regular intervals on the periphery around a central axis (the longitudinal axis Ax of the insertion part 22) of the distal end hard part 40. The respective ultrasound oscillators 48 are sequentially driven based on a drive signal input from the ultrasound processor device 14 (see FIG. 1). Thus, radial electronic scanning is performed with a range in which the ultrasound oscillators 48 are arranged, as a scanning range.

The ultrasound transducer 46 comprises a plurality of individual electrodes corresponding to a plurality of ultrasound oscillators 48 and a common electrode common to a plurality of ultrasound oscillators 48. The ultrasound transducer 46 comprises a flexible print substrate 73 to which each of the common electrode and a plurality of individual electrodes is connected.

The ultrasound transducer 46 has an acoustic matching layer (not shown) laminated on an outer peripheral surface side of the ultrasound oscillators 48, an acoustic lens 49 laminated on an outer peripheral surface side, and a backing material layer (not shown) laminated on an inner peripheral surface side of the ultrasound oscillators 48.

As shown in FIG. 3, the flexible print substrate 73 that is attached to a side surface on a proximal end side of the backing material layer (not shown) is electrically connected to a plurality of individual electrodes of the ultrasound oscillators 48 at one end and is wired and connected to a plurality of signal lines 74 of the ultrasonic cable 72 at the other end. The ultrasonic cable 72 is held inside the distal end hard part 40 by the bracket 80. The signal lines 74 may be configured with, for example, a coaxial cable.

In the distal end hard part 40 shown in FIG. 2, a balloon (not shown) into which an ultrasonic wave transmission medium (for example, water or oil) covering the ultrasonic observation part 36 is injected may be attachably and detachably mounted.

As shown in FIG. 3, in the ultrasonic endoscope 12 comprising the radial type ultrasound transducer 46, the ultrasound transducer 46 is disposed on the outer peripheral surface of the distal end hard part 40.

As shown in FIG. 3, a portion where the ultrasound transducer 46 is disposed constitutes an outer peripheral surface portion 56 in the distal end surface 51 on a distal end side in the longitudinal axis Ax direction. The outer peripheral surface portion 56 becomes a dead space in the distal end surface 51 because the observation window 62, the illumination windows 64, the nozzle 66, and the forceps port 60 constituting the functional units of the ultrasonic endoscope 12 cannot be disposed. For this reason, the observation window 62, the illumination windows 64, the nozzle 66, and the forceps port 60 are provided with bias to the center surface portion 54 on the center side of the distal end surface 51 excluding the outer peripheral surface portion 56.

Accordingly, the inventors have conducted intensive studies and have completed the ultrasonic endoscope 12 capable of improving water droplet removal performance using the outer peripheral surface portion 56 that becomes a dead space in the distal end surface 51.

### First Embodiment

The configuration of the distal end surface 51 of the distal end hard part 40 that is a characteristic feature of the present invention will be described.

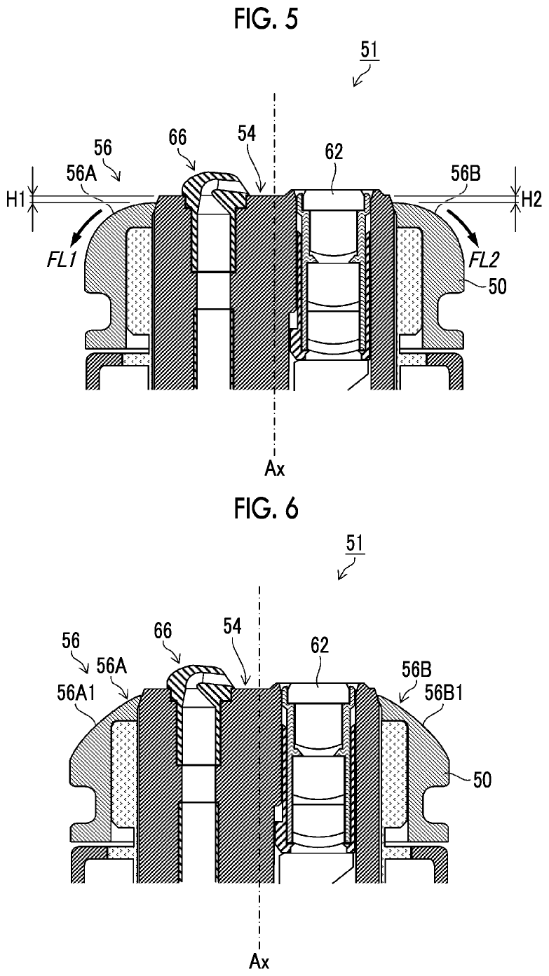

FIG. 4 is an enlarged plan view of the distal end surface 51 of the distal end hard part 40 as viewed from a vertical direction to the distal end surface 51. FIG. 5 is a sectional view taken along the line 5-5 of FIG. 4.

As shown in FIG. 4, the distal end surface 51 includes the center surface portion 54 and the outer peripheral surface portion 56. The center surface portion 54 is positioned on the center side in the distal end surface 51, and the outer peripheral surface portion 56 is positioned outside the center surface portion 54 in the distal end surface 51 and has an annular shape surrounding the center surface portion 54. That is, the center surface portion 54 is positioned on the center side of the distal end surface 51 based on relative comparison with the outer peripheral surface portion 56.

The center surface portion 54 has a structure based on a substantially circular flat surface around a position crossing the longitudinal axis Ax in plan view. In the center surface portion 54, the forceps port 60, the observation window 62, the illumination windows 64, and the nozzle 66 are provided. The center surface portion 54 is a region that includes all of the forceps port 60, the observation window 62, the illumination windows 64, and the nozzle 66 and has a substantially circular shape around the longitudinal axis Ax. The substantially circular shape includes shapes, such as a circular shape, an elliptical shape, and an oval shape.

The center surface portion 54 is made of, for example, a metal material, such as stainless steel. A plurality of through-holes are formed in the center surface portion 54, and the forceps port 60, the observation window 62, the illumination windows 64, and the nozzle 66 are held by the through-holes. The outer peripheral surface portion 56 is configured with the resin distal end cap 50 that is a separate member from the center surface portion 54. Note that the materials of the center surface portion 54 and the outer peripheral surface portion 56 are not particularly limited.

In the center surface portion 54, the observation window 62 and the two illumination windows 64 that constitute the endoscope observation part 38 are disposed. The two illumination windows 64 are disposed adjacent to each other at positions on both sides of the observation window 62 with the observation window 62 interposed therebetween. As described above, the observation window 62 acquires the endoscope image of the part to be observed, the illumination windows 64 irradiate the part to be observed with the illumination light.

In the center surface portion 54, the forceps port 60 is disposed at a distance from the observation window 62. The forceps port 60 is a region having a greatest area in the center surface portion 54, and is disposed at a position close to the longitudinal axis Ax with respect to the observation window 62. As described above, the forceps port 60 functions as the treatment tool outlet port and the suction port.

In the center surface portion 54, the nozzle 66 is disposed at a position between the forceps port 60 and one illumination window 64. The nozzle 66 comprises a jetting port 66A, and the jetting port 66A is directed toward the observation window 62. The jetting port 66A of the nozzle 66 jets a fluid (liquid or gas) to the surface of the observation window 62 and a peripheral portion (observation window 62 and the like) and blows off and removes dirt attached to the observation window 62.

Note that, as shown in FIG. 4, the distal end surface 51 has the outer peripheral surface portion 56 that is a dead space caused by the ultrasound transducer 46, outside the center surface portion 54. For this reason, water droplet or dirt (water droplet or the like) does not fly out with the fluid from the nozzle 66 and remains attached to the outer peripheral surface portion 56. In this case, water droplet or the like attached to the outer peripheral surface portion 56 easily returns to the observation window 62 and the like. Water droplet or the like attached to the outer peripheral surface portion 56 is caught during air supply from the nozzle 66 and flows into and easily returns to the observation window 62 and the like. Such phenomenon results in degradation of water droplet removal performance.

Accordingly, the inventors have focused that at least part of region of the outer peripheral surface portion 56 is highly likely to influence degradation of the water droplet removal performance of the observation window 62 and the like, and have found that at least part of region in the outer peripheral surface portion 56 is made to have a height different from the center surface portion 54 to promote a water droplet removal effect. In the present specification, the longitudinal axis Ax direction of the insertion part 22 is defined as a height direction of the distal end surface 51. For example, with the center surface portion 54 as a reference, in a case where a region is positioned on the proximal end side with respect to the center surface portion 54, this means that the region has a height lower than the center surface portion 54, and in a case where a region is positioned on the distal end side with respect to the center surface portion 54, this means that the region has a height higher than the center surface portion 54. The reference of the center surface portion 54 is a region in the center surface portion 54 where the forceps port 60, the observation window 62, the illumination windows 64, and the nozzle 66 are not provided.

Next, a nozzle adjacent region 56A and an observation window adjacent region 56B that are at least part of region in the outer peripheral surface portion 56 will be described. In FIG. 4, for ease of understanding, colors of the nozzle adjacent region 56A and the observation window adjacent region 56B in the outer peripheral surface portion 56 are changed.

Nozzle Adjacent Region

As shown in FIG. 4, the outer peripheral surface portion 56 includes the nozzle adjacent region 56A adjacent to the nozzle 66.

The nozzle adjacent region 56A of the embodiment includes at least a region A indicated by an arrow. The region A includes, for example, a region from an extending position (virtual straight line L1) of the jetting port 66A of the nozzle 66 on a side opposite to the forceps port 60 in a width direction to a position (virtual straight line L2) of the nozzle 66 on a side opposite to the observation window 62. The nozzle adjacent region 56A may be greater than the region A. The virtual straight line L2 is a straight line that connects the center of the observation window 62 and the center of the jetting port 66A of the nozzle 66, and is a straight line extending to a side opposite to the observation window 62. The center of the jetting port 66A is a position where a length of the jetting port 66A becomes half in plan view.

By the way, water droplet or the like that remains in a region of the outer peripheral surface portion 56 adjacent to the nozzle 66 is easily caught during air supply from the nozzle 66 and easily returns to the observation window 62 and the like.

Accordingly, the nozzle adjacent region 56A has a height lower than the center surface portion 54 by H1 as shown in FIG. 5. The nozzle adjacent region 56A corresponds to an example of at least part of region in an outer peripheral surface portion of the present invention. Because the height of the nozzle adjacent region 56A is lower by H1, water droplet or the like easily flows down from the nozzle adjacent region 56A in a liquid flow direction FL1 (FIGS. 4 and 5), and as a result, water droplet or the like is difficult to be caught into air during air supply from the nozzle 66, and water droplet or the like is difficult to return to the observation window 62.

Observation Window Adjacent Region

As shown in FIG. 4, the outer peripheral surface portion 56 includes the observation window adjacent region 56B adjacent to the observation window 62.

The observation window adjacent region 56B of the embodiment is positioned on a side opposite to the nozzle 66 with respect to the observation window 62. The observation window adjacent region 56B includes at least a region B indicated by an arrow, and the region B is, for example, a region including a jetting range of a liquid jetted from the nozzle 66. The region B is preferably a range between two virtual tangent lines L3 and L4 in contact with an outer periphery of the observation window 62 with the center of the jetting port 66A as a starting point. The observation window adjacent region 56B may be greater than the region B.

By the way, water droplet or the like that remains in a region of the outer peripheral surface portion 56 adjacent to the observation window 62 easily returns to the observation window 62 and the like. Accordingly, the observation window adjacent region 56B has a height lower than the center surface portion 54 by H2 as shown in FIG. 5. The observation window adjacent region 56B is an example of at least part of region in an outer peripheral surface portion of the present invention. Because the height of the observation window adjacent region 56B is lower by H2, water droplet or the like easily flows down from the observation window adjacent region 56B in a liquid flow direction FL2 (FIGS. 4 and 5), and as a result, water droplet or the like is difficult to return to the observation window 62 and the like.

According to the first embodiment, because the outer peripheral surface portion 56 of the distal end surface 51 comprises the nozzle adjacent region 56A and the observation window adjacent region 56B having a height lower than the center surface portion 54, water droplet or the like is difficult to return to the observation window 62 and the like, and it is possible to improve water droplet removal performance with respect to the observation window 62 and the like. According to the first embodiment, it is possible to effectively use the outer peripheral surface portion 56 that becomes a dead space.

### Preferred Modification Example 1

Although a case where the outer peripheral surface portion 56 comprises the nozzle adjacent region 56A and the observation window adjacent region 56B has been described, preferred Modification Example 1 of the first embodiment. In preferred Modification Example 1, a characteristic where water droplet or the like is difficult to remain, for example, surface wettability or a chamfer structure is given to the nozzle adjacent region 56A and the observation window adjacent region 56B.

Surface Wettability

It is preferable that the surface wettability of the center surface portion 54 is different from the surface wettability of the nozzle adjacent region 56A and the observation window adjacent region 56B. The surface wettability is easiness of attachment of a liquid to a solid.

For example, the surface wettability of the center surface portion 54 can be made hydrophilic, and the surface wettability of the nozzle adjacent region 56A and the observation window adjacent region 56B can be made hydrophobic. In this case, water droplet or the like easily gather to the center surface portion 54, and water droplet or the like of the center surface portion 54 is easily sucked from the forceps port 60. As a result, water droplet or the like is more reliably removed from the observation window 62 and the like.

The surface wettability of the center surface portion 54 can be made hydrophobic, and the surface wettability of the nozzle adjacent region 56A and the observation window adjacent region 56B can be made hydrophilic. In this case, it is possible to make water droplet from the center surface portion 54 be easily removed. It is possible to make water droplet or the like difficult to remain on the observation window 62 and the like.

The surface wettability can be changed by changing the surface physical properties of the center surface portion 54, the nozzle adjacent region 56A, and the observation window adjacent region 56B. Examples of changing the surface physical property include methods, such as changing materials, changing surface roughness, and changing surface coating films. Determination regarding whether the surface wettability is hydrophobic or hydrophilic can be performed based on a water contact angle or the like.

Chamfer Structure

It is preferable that a chamfer structure is provided in outer peripheral end portions of the nozzle adjacent region 56A and the observation window adjacent region 56B. FIG. 6 is a partial sectional view of the distal end hard part 40 at the same position as in FIG. 5.

As shown in FIG. 6, a chamfer structure 56A1 is provided in the nozzle adjacent region 56A. The chamfer structure 56A1 is provided, whereby water droplet or the like is easily removed from the nozzle adjacent region 56A. Similarly, a chamfer structure 56B1 is provided, whereby water droplet or the like is easily removed from the observation window adjacent region 56B. The chamfer structures 56A1 and 56B1 are configured with inclined surfaces in which a diameter expands from the distal end side toward the proximal end side in the outer peripheral end portion of the outer peripheral surface portion 56. The inclined surface may be not only curved surfaces but also flat surfaces.

The chamfer structure and the surface wettability are combined, so that water droplet or the like can be made to be more easily removed from the nozzle adjacent region 56A and the observation window adjacent region 56B.

### Preferred Modification Example 2

Although a case where the outer peripheral surface portion 56 comprises the nozzle adjacent region 56A and the observation window adjacent region 56B has been described, a region of the outer peripheral surface portion 56 other than the nozzle adjacent region 56A and the observation window adjacent region 56B is made to have a height higher than the center surface portion 54, so that the observation window 62 can be protected.

As shown in FIG. 4, the outer peripheral surface portion 56 comprises two outer peripheral surface portion constitution regions 56C other than the nozzle adjacent region 56A and the observation window adjacent region 56B. Each of the two outer peripheral surface portion constitution regions 56C is positioned between the nozzle adjacent region 56A and the observation window adjacent region 56B.

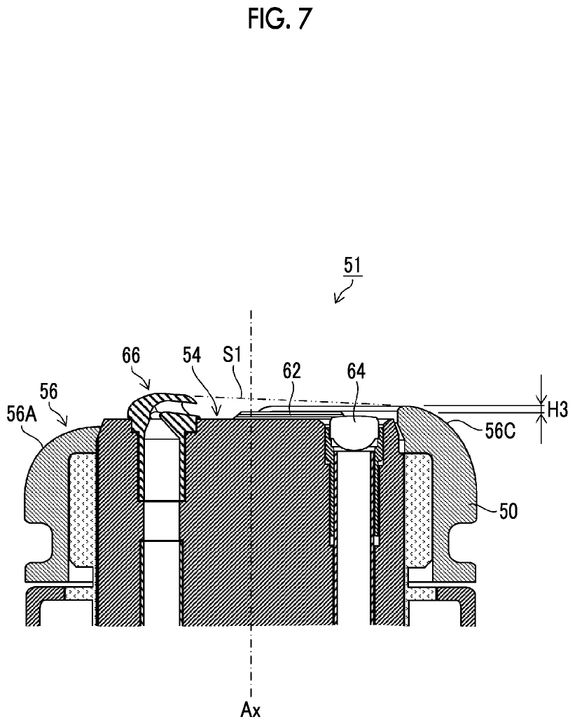

As shown in FIG. 7, the outer peripheral surface portion constitution regions 56C have a height higher than the center surface portion 54 by H3. Here, as shown in FIG. 4, a plane defined by three points including the distal end of the nozzle 66 and any two points on the two outer peripheral surface portion constitution regions 56C, is referred to as a virtual plane S1. In this case, as shown in FIGS. 4 and 7, the observation window 62 is disposed in the virtual plane S1, and the virtual plane S1 is positioned on the distal end side with respect to the observation window 62. In a case where the virtual plane S1 satisfies such conditions, it is possible to prevent a wall, a floor, or the like from being brought into contact with the observation window 62 with the nozzle 66 and the two outer peripheral surface portion constitution regions 56C.

Because the two outer peripheral surface portion constitution regions 56C have a height higher than the center surface portion 54 by H3, the two outer peripheral surface portion constitution regions 56C correspond to an example of at least part of region in an outer peripheral surface portion of the present invention. According to preferred Modification Example 2 of the first embodiment, the outer peripheral surface portion 56 that becomes a dead space can be effectively used for the protection of the observation window 62.

### Second Embodiment

Next, a second embodiment will be described. In the above-described first embodiment, the outer peripheral surface portion 56 comprises the nozzle adjacent region 56A and the observation window adjacent region 56B having a height lower than the center surface portion 54. In contrast, in the second embodiment, the outer peripheral surface portion 56 comprises the nozzle adjacent region 56A having a height lower than the center surface portion 54, and the observation window adjacent region 56D having a height higher than the center surface portion 54. Hereinafter, description of the points common to the first embodiment will not be repeated, and points different from the first embodiment will be described.

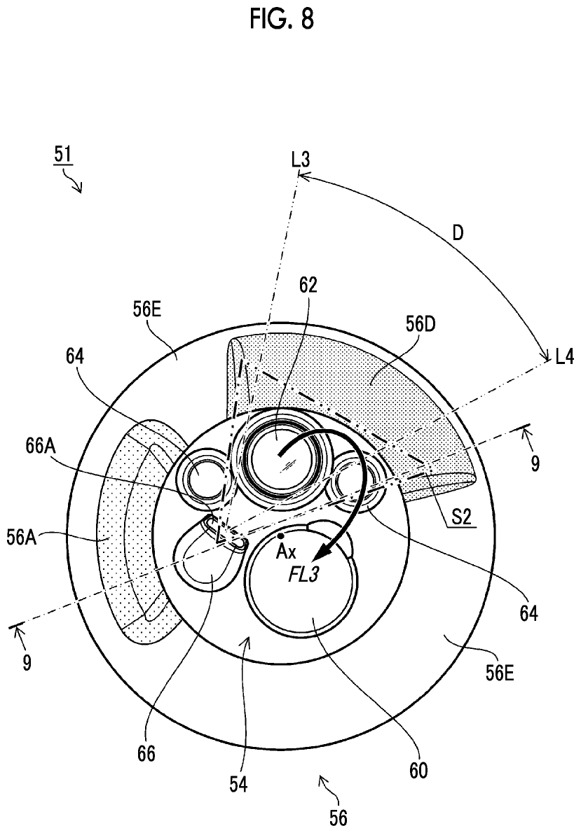

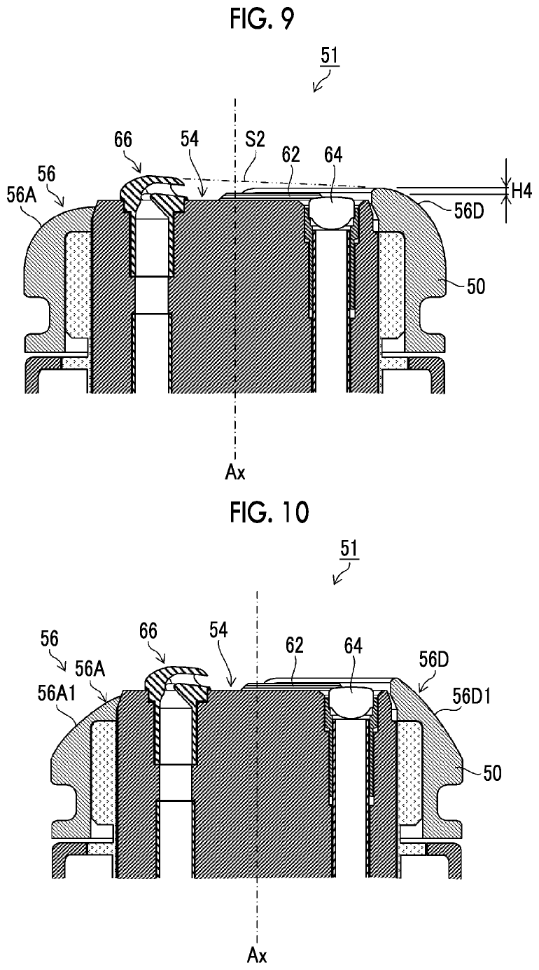

FIG. 8 is an enlarged view of a distal end surface as viewed from a vertical direction to the distal end surface of the second embodiment. FIG. 9 is a partial sectional view of a distal end hard part taken along the line 8-8 of FIG. 8.

As shown in FIG. 8, the distal end surface 51 comprises the center surface portion 54 and the outer peripheral surface portion 56. In the center surface portion 54, the forceps port 60, the observation window 62, the illumination windows 64, and the nozzle 66 are provided. The outer peripheral surface portion 56 includes the nozzle adjacent region 56A having a height lower than the center surface portion 54. In regard to such points, the second embodiment is common to the first embodiment.

Observation Window Adjacent Region

As shown in FIG. 8, the outer peripheral surface portion 56 includes an observation window adjacent region 56D adjacent to the observation window 62.

The observation window adjacent region 56D of the embodiment is positioned on a side opposite to the nozzle 66 with respect to the observation window 62. The observation window adjacent region 56D includes at least a region D indicated by an arrow. The region D is, for example, a region including a jetting range of a liquid jetted from the nozzle 66. The region D is preferably a range between the two virtual tangent lines L3 and L4 in contact with the outer periphery of the observation window 62 with the center of the jetting port 66A as a starting point. While the region D is basically the same as the region B of the first embodiment, as shown in FIG. 9, the observation window adjacent region 56D of the second embodiment has a height than the center surface portion 54 by H4. The observation window adjacent region 56D is an example of at least part of region in an outer peripheral surface portion of the present invention. Because the height of the observation window adjacent region 56D is higher by H4, the observation window adjacent region 56D becomes a step that protrudes in the height direction with respect to the center surface portion 54. Because the step becomes a wall of a liquid jetted from the nozzle 66, water droplet or the like blown off by a fluid falls on the observation window adjacent region 56D. Then, water droplet or the like is guided to the forceps port 60 through the observation window adjacent region 56D as indicated by a liquid flow direction FL3. As a result, water droplet or the like is more reliably sucked from the forceps port 60. Water droplet or the like is difficult to remain in the observation window adjacent region 56D.

According to the second embodiment, because the outer peripheral surface portion 56 of the distal end surface 51 comprises the nozzle adjacent region 56A having a height lower than the center surface portion 54 and the observation window adjacent region 56D having a height higher than the center surface portion 54, water droplet or the like is difficult to return to the observation window 62 and the like, and water droplet or the like is more reliably sucked from the forceps port 60. Thus, it is possible to improve water droplet removal performance. According to the second embodiment, the outer peripheral surface portion 56 that becomes a dead space can be effectively used.

### Preferred Modification Example 1

While a case where the outer peripheral surface portion 56 comprises the nozzle adjacent region 56A and the observation window adjacent region 56D has been described, preferred Modification Example 1 of the second embodiment will be described. As in the first embodiment, in preferred Modification Example 1 of the second embodiment, a characteristic where water droplet or the like is difficult to remain in the nozzle adjacent region 56A and the observation window adjacent region 56D, such as the surface wettability and the chamfer structure, can be given.

In regard to the surface wettability, it is preferable that the surface wettability of the center surface portion 54 is different from the surface wettability of the nozzle adjacent region 56A and the observation window adjacent region 56D. As in the first embodiment, the surface wettability of the center surface portion 54 can be made hydrophilic, and the surface wettability of the nozzle adjacent region 56A and the observation window adjacent region 56D can be made hydrophobic. The surface wettability of the center surface portion 54 can be made hydrophobic, and the surface wettability of the nozzle adjacent region 56A and the observation window adjacent region 56D can be made hydrophilic. In the second embodiment, it is also possible to obtain the same effects as in the first embodiment.

In regard to the chamfer structure, as shown in FIG. 10, a chamfer structure 56A1 and a chamfer structure 56D1 are provided in outer peripheral end portions of the nozzle adjacent region 56A and the observation window adjacent region 56D, respectively. The chamfer structure 56A1 and the chamfer structure 56D1 are provided, whereby, in the second embodiment, as in the first embodiment, water droplet or the like is easily removed from the nozzle adjacent region 56A and the observation window adjacent region 56D. FIG. 10 is a partial sectional view of the distal end hard part 40 at the same position as in FIG. 9.

### Preferred Modification Example 2

While a case where the outer peripheral surface portion 56 comprises the nozzle adjacent region 56A and the observation window adjacent region 56D has been described, in addition, it is possible to protect the observation window 62 using the nozzle adjacent region 56A and the observation window adjacent region 56D of the outer peripheral surface portion 56.

As shown in FIG. 9, the observation window adjacent region 56D has a height higher than the center surface portion 54 by H4. Here, as shown in FIG. 8, a plane with the distal end of the nozzle 66 and any two points of the observation window adjacent region 56D as apexes is referred to as a virtual plane S2. In this case, as shown in FIGS. 8 and 9, the observation window 62 is disposed in the virtual plane S2, and the virtual plane S2 is positioned on the distal end side with respect to the observation window 62. In a case where the virtual plane S2 satisfies such conditions, it is possible to prevent a wall, a floor, or the like from being brought into contact with the observation window 62 with the nozzle 66 and the observation window adjacent region 56D.

According to preferred Modification Example 2 of the second embodiment, the outer peripheral surface portion 56 that becomes a dead space can be effectively used for the protection of the observation window 62.

As shown in FIG. 8, the outer peripheral surface portion 56 comprises two outer peripheral surface portion constitution regions 56E other than the nozzle adjacent region 56A and the observation window adjacent region 56D. Each of the two outer peripheral surface portion constitution regions 56E is positioned between the nozzle adjacent region 56A and the observation window adjacent region 56B. The two outer peripheral surface portion constitution regions 56E have the same height as the center surface portion 54.

As described above, in the first embodiment, a case where the outer peripheral surface portion 56 comprises the nozzle adjacent region 56A and the observation window adjacent region 56B has been illustrated, and in the second embodiment, a case where the outer peripheral surface portion 56 comprises the nozzle adjacent region 56A and the observation window adjacent region 56D has been illustrated. Note that the present invention is not limited thereto, and only one may be provided in the outer peripheral surface portion 56. According to the present invention, some improvements or modifications may be made without departing from the spirit and scope of the present invention.

### EXPLANATION OF REFERENCES

10: ultrasonography system
12: ultrasonic endoscope
14: ultrasound processor device
16: endoscope processor device
18: light source device
20: monitor
21a: water supply tank
21b: suction pump
22: insertion part
24: operating part
26: universal cord
28a: air/water supply button
28b: suction button
29: angle knob
30: treatment tool insertion port
32a: connector
32b: connector
32c: connector
34a: air/water supply tube
34b: suction tube
36: ultrasonic observation part
38: endoscope observation part
40: distal end hard part
42: bendable part
44: soft part
46: ultrasound transducer
48: ultrasound oscillator
49: acoustic lens
50: distal end cap
51: distal end surface
52: proximal end-side ring
53: base member
54: center surface portion
56: outer peripheral surface portion
56A: nozzle adjacent region
56A1: chamfer structure
56B: observation window adjacent region
56B1: chamfer structure
56C: outer peripheral surface portion constitution region
56D: observation window adjacent region
56D1: chamfer structure
56E: outer peripheral surface portion constitution region
60: forceps port
62: observation window
64: illumination window
66: nozzle
66A: jetting port
68: air/water supply channel
70: light guide
72: ultrasonic cable
73: flexible print substrate
74: signal line
80: bracket
84: forceps tube
86: lens group
88: prism
90: imaging element
92: substrate
94: signal line
Ax: longitudinal axis
FL1: liquid flow direction
FL2: liquid flow direction
FL3: liquid flow direction
L1: virtual straight line
L2: virtual straight line
L3: virtual tangent line
L4: virtual tangent line
S1: virtual plane
S2: virtual plane

What is claimed is:

1. An ultrasonic endoscope comprising:
a radial type ultrasound transducer disposed in a distal end part of an elongated insertion part to be inserted into a subject,
wherein a distal end surface of the distal end part includes, in a case of being viewed from a direction perpendicular to the distal end surface, a center surface portion positioned on a center side of the distal end surface, and an annular outer peripheral surface portion positioned outside the center surface portion,
an observation window, an illumination window, a forceps port, and a nozzle are provided in the center surface portion,
the outer peripheral surface portion is a dead space of the distal end surface caused by the ultrasound transducer, and
in a case where a longitudinal axis direction of the insertion part is defined as a height direction on the distal end surface,
at least part of region of the outer peripheral surface portion has a height different from the center surface portion.

2. The ultrasonic endoscope according to claim 1,
wherein the at least part of region includes a nozzle adjacent region adjacent to the nozzle.

3. The ultrasonic endoscope according to claim 2,
wherein the nozzle adjacent region has a height lower than the center surface portion.

4. The ultrasonic endoscope according to claim 2,
wherein the nozzle adjacent region includes a region from an extending position of a jetting port of the nozzle to a position of the nozzle on a side opposite to the observation window.

5. The ultrasonic endoscope according to claim 1,
wherein the at least part of region includes an observation window adjacent region adjacent to the observation window.

6. The ultrasonic endoscope according to claim 5,
wherein the observation window adjacent region has a height lower than the center surface portion.

7. The ultrasonic endoscope according to claim 5,
wherein the observation window adjacent region has a height higher than the center surface portion.

8. The ultrasonic endoscope according to claim 5,
wherein the observation window adjacent region is positioned on a side opposite to the nozzle with respect to the observation window.

9. The ultrasonic endoscope according to claim 8,
wherein the observation window adjacent region is a region including a jetting range of a liquid jetted from the nozzle.

10. The ultrasonic endoscope according to claim 8,
wherein the observation window adjacent region is a region including a range sandwiched between two virtual tangent lines in contact with an outer periphery of the observation window with a center of the jetting port of the nozzle as a starting point.

11. The ultrasonic endoscope according to claim 1,
wherein the at least part of region includes a nozzle adjacent region adjacent to the nozzle and an observation window adjacent region adjacent to the observation window.

12. The ultrasonic endoscope according to claim 1,
wherein the at least part of region is different in surface wettability from the center surface portion.

13. The ultrasonic endoscope according to claim 1,
wherein the outer peripheral surface portion has a chamfer structure in an outer peripheral end portion.

14. The ultrasonic endoscope according to claim 1,
wherein the outer peripheral surface portion is configured with a distal end cap that is a separate member from the center surface portion.
